(19) United States
(12) Patent Application Publication
(10) Pub. No.: US 2008/0058703 A1
(43) Pub. Date: Mar. 6, 2008

(54) Drug electrotransport with hydration measurement of hydratable reservoir

(76) Inventors: Janardhanan A. Subramony, Santa Clara, CA (US); Michel J.N. Cormier, Mountain View, CA (US); Rama V. Padmanabhan, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/893,732

(22) Filed: Aug. 17, 2007

Related U.S. Application Data
(60) Provisional application No. 60/841,092, filed on Aug. 29, 2006.

(51) Int. Cl. A61N 1/30

(52) U.S. Cl. 604/20

(57) ABSTRACT

A transdermal electrotransport drug delivery system with hydratable reservoir and method for drug delivery to an individual. The system has a hydratable reservoir with impedance measurement means for determining level of hydration in the reservoir.

### CROSS REFERENCE TO RELATED U.S. APPLICATION DATA.

The present application claims the priority benefit of provisional application 60/841,092, filed Aug. 29, 2006, which is incorporated by reference herein.

### TECHNICAL FIELD

This invention relates to a medical device for electrotransport transdermal administration of a drug and to a method of treating a subject by administering a drug to a patient with the medical device by electrotransport. In particular, the invention relates to transdermal electrotransport systems for administration of a drug with a hydratable drug reservoir.

### BACKGROUND

The natural barrier function of the body surface, such as skin, presents a challenge to delivery of therapeutics into blood circulation in the body. Transdermal devices for the delivery of biologically active agents or drugs have been used for maintaining health and treating therapeutically a wide variety of ailments. For example, analgesics, steroids, etc., have been delivered with such devices. Transdermal drug delivery can generally be considered to belong to one of two groups: transport by a “passive” mechanism or by an “active” transport mechanism. In the former embodiment, such as drug delivery skin patches, the drug is incorporated in a solid matrix, a reservoir, and/or an adhesive system.

Most passive transdermal delivery systems are not capable of delivering drugs under a specific profile, such as by ‘on-off’ mode, pulsatile mode, etc. Consequently, a number of alternatives have been proposed where various forms of energy drive the flux of the drug(s). Some examples include the use of iontophoresis, ultrasound, electroporation, heat and microneedles. These are considered to be “active” delivery systems. Iontophoresis, for example, is an “active” delivery technique that transports solubilized drugs across the skin by an electrical current. The feasibility of this mechanism is constrained by the solubility, diffusion and stability of the drugs, as well as electrochemistry in the device.

A significant advantage of active transdermal technologies is that the timing and profile of drug delivery can be controlled, so that doses may be automatically controlled on a pre-determined schedule or self-delivered by the patient based on need. For example, U.S. Pat. Nos. 5,057,072; 5,084,008; 5,147,297; 6,039,977; 6,049,733; 6,181,963, 6,216,033, 6,317,629, and US Patent Publication 20030191946, are related to electrotransport transdermal delivery of drugs. Also, electrotransport systems that additionally use microprotrusion array for assisting therapeutic agent delivery have also been disclosed in U.S. Patent Publication 20020016562.

In iontophoretic systems, one electrode, called the active or donor electrode, is the electrode from which the active agent is delivered into the body. The other electrode, called the counter or return electrode, serves to close, i.e., complete, the electrical path (circuit) through the body. In conjunction with the patient's body tissue, e.g., skin, the circuit is closed by connection of the electrodes to a source of electrical energy, and usually to circuitry capable of controlling the current passing through the device. If the ionic substance to be driven into the body is positively charged, then the positive electrode (the anode) will be the active (or donor) electrode and the negative electrode (the cathode) will serve as the counter electrode. If the ionic substance to be delivered is negatively charged, then the cathodic electrode will be the active (or donor) electrode and the anodic electrode will be the counter electrode. Electrotransport devices require a reservoir or source of the active agent that is to be delivered or introduced into the body. Such reservoirs are connected to the anode or the cathode of the electrotransport device to provide a fixed or renewable source of one or more desired active agents.

Although electrotransport is useful for delivery of ionic drugs, not all ionic drugs are suitable for such delivery. Drug stability, both in use and during storage, is important for the manufacture and storage of pharmaceutical products. It is essential to find a formulation that will provide acceptable stability for the active pharmaceutical ingredient for a period of storage, such as the recommended period before the expiration of which the drug should be used (shelf life). A drug cannot be incorporated into a product if the drug molecule is not stable in the product formulation. Thus, many drugs, although therapeutically useful and feasible to be delivered transdermally, would not be available to patients without ways to maintain the stability over a period of time adequate for distribution through commercial channels and use.

Yet another challenge to achieve practical electrotransport delivery involves maintaining physical compatibility of moisture-sensitive electrical components present within the delivery system with water-based formulations in close proximity. Metallic components of the sensitive electrical circuitry, for example, can be subject to breakdown by corrosion if exposed to humidity or bulk water of aqueous-type formulations. Keeping the formulation in the dry or dehydrated state until just prior to use would promote stability of the dosage form during storage.

Drug reservoirs used in iontophoresis are typically aqueous based systems using hydrophilic polymers. This allows for maximum ion mobility and conductivity under the influence of an electric field. There are a large variety of drug reservoirs in the literature to date, such as polyvinyl alcohol (PVOH), as well as cellulose-based polymers. Most reservoirs contain drug salt dissolved in a solution. This form offers the simplest means of drug loading, yet in prior methods and devices, the problem of solution (e.g., aqueous drug formulation) and electrical stability has not been adequately addressed.

Attempts to solve the lack of aqueous stability of drugs within reservoirs include the use of hydratable systems. Hydration, as used herein, refers to the absorption of any solvent or agent into the hydratable reservoir so as to provide a liquid medium for ion movement, e.g., charged drug molecules in ionic form for electrotransport application. Aqueous drug solution is, of course, one example of such a liquid medium. In a hydrated reservoir, positive ions and negative ions can move under electromotive force in the appropriate direction toward or away from electrodes according to their respective polarities. Examples of systems that have been developed in which the drug-containing reservoir is hydrated prior to use are polyurethane based systems. Examples of prior disclosures on hydration of reservoirs include, for example, U.S. Pat. Nos. 5,236,412; 5,288,289; 5,533,972; 5,582,587; 5,645,527; 6,275,728; and 6,317,629, the disclosure of which are incorporated by reference in their entireties.

However, slow hydration kinetics, long solvation times, and the difficulty of determining whether a reservoir has been adequately hydrated are some of the problems associated with hydratable systems. If a reservoir is not hydrated adequately ionic movement will be hindered and drug delivery will be ineffective. Poor or incomplete hydration of the drug reservoir is likely to result in poor skin contact resulting in preferential transport pathways with low resistance within the application site. This in turn will result in focal irritation due to high current density within current pathways on the application site. On the other hand, overhydration is also undesirable in that it may adversely affect the drug stability and mechanical property of the gel in the reservoir. Further, waiting for a long time as a conservative approach to allow for a system to hydrate is inconvenient and deters acceptance of the system. Hydration kinetics are traditionally measured by immersing polymer reservoirs in distilled water and monitoring weight change as a function of time in seconds. Such a method is, of course, impractical if the reservoir is to be used on a patient after hydration or if the measurement of hydration is to be done in situ. In situ hydration is more desirable because of the reservoir is small in size and excessive manipulation to install a gel might damage the delicate gel.

Although the transdermal delivery of therapeutic agents has been the subject of intense research and development for over 30 years, because of the above reasons, thus far only a few drugs have been found to be suitable for transdermal electrotransport application. Further improvements are needed for better systems for hydrating iontophoretic drug delivery systems. The present invention provides methodology and devices with which hydration process can be better controlled, thus providing more reliable electrotransport drug delivery.

### SUMMARY

Drug ion migration requires the presence of a certain level of polar liquid. Drug flux, the amount of mass transported across a membrane per unit area, time, and current, is a function of the hydration condition of the reservoir that contains the drug. The resistance or conductivity value of a hydratable drug-containing reservoir is a function of its hydration state and indicative of the nature and capability of ion transport in the system. Because conductivity or impedance can be correlated to the degree of hydration, the present invention takes advantage of the fact that the impedance of a reservoir, whether a hydratable reservoir before hydration or after hydration (e.g., a gel layer or layers of films), can be measured to determine the hydration level of the reservoir, thereby allowing electrotransport to begin only when an adequate level of hydration has been achieved.

Although attempts were made to measure impedance on skin before for various reasons (see, e.g., U.S. Pat. Nos. 5,003,987; 5,289,822; 6,167,301; 6,391,015; 6,731,987; and 20030204163), thus far no one has disclosed a device or a method of monitoring impedance across a reservoir of an electrotransport device to determine the progress of hydration in an electrotransport reservoir. In one aspect, having the mechanism to monitor impedance across the reservoir and the impedance from the reservoir to the skin provides a reliable, stable, compact system that benefits individuals in need of electrotransport drug delivery.

This invention provides methodology and devices for improving iontophoretic drug delivery with systems having hydratable reservoirs. In one aspect, a system is provided to have an impedance sensor for determining that an adequate level of hydration has taken place. In another aspect, the system, by determining the impedance of the reservoir, allows iontophoretic drug delivery to commence when a desirable level of impedance has been reached (i.e., the impedance has fallen to or below a predetermined level).

In one aspect, an iontophoretic drug delivery system has a controller controlling current flow from a reservoir (e.g., donor reservoir) to the body surface and the controller is designed and constructed to send a test current across the reservoir to determine the impedance thereof. The controller will allow a drug delivery current flow to be switched on only after the impedance across the reservoir has fallen below a predetermined condition (e.g., a threshold level) as the reservoir undergoes hydration.

The invention provides a method and system to monitor the degree of hydration of the reservoir gel in an in-situ fashion from conductivity/impedance measurements across the reservoir of interest (say, the donor reservoir). One of the advantages of such methods and systems is that the hydration of the hydratable reservoir can be gauged without having to take the reservoir from the donor compartment. In one aspect, resistance can be measured under direct current (DC). In yet another aspect, system and method are provided that alternating current (AC) impedance measurements are done to provide information on the extent of hydration. This approach is particularly suitable for indicating condition of long range ion transport because using alternating current does not lead to concentration polarization.

In another aspect, a kit including a portable electrotransport device with dehydrated reservoir and a hydrating liquid source can be provided. The portable electrotransport device can include an impedance meter or is connectable to a separate impedance meter.

Conductivity measurements can be used to indicate ion transport in a system, which depends on the mobility of ions. Aqueous solutions containing ions and water would facilitate ion transport through a reservoir, e.g., a hydrogel. In this case, liquid electrolytes containing ions are strong conductors of current due to the ion mobility in the aqueous medium. In the case of solid electrolytes containing an excess of ionic charge in a solid substrate, defects in the crystal structure (such as Schottky, Frenkel, and interstitial) and hydration can help facilitate ion transport. The same applies to non-aqueous gel electrolytes where salvation by an organic solvent or the presence of any hydrophilic components can contribute to the conductivity. Thus, impedance measurement to determine the extent of hydration (meaning solvation using organic solvent in this case) can also be accomplished in devices having non-aqueous gels. Certain types of solid electrolytes include polymer electrolytes, in which transport of ions is believed to be due to low amplitude segmental motion of the polymer under an applied electric field. It is contemplated that the present invention is applicable in all such hydration determination (which may be salvation with water, aqueous, or organic solvents).

In one aspect, the present invention provides a method of preparing an iontophretic drug delivery device. The method includes the steps of hydrating a hydratable reservoir in an iontophretic drug delivery device by providing a liquid to the hydratable reservoir, sensing impedance across the hydratable reservoir, monitoring the impedance until the impedance has reached a predetermined condition, and refraining from providing more of the liquid to the hydratable reservoir after the impedance has reached a predetermined condition.

In one aspect, the present invention provides a method of preparing an electrotransport device for drug delivery, including forming a hydratable reservoir matrix in the device and providing impedance measurement capability. The method includes providing a prehydration device comprising a pair of electrode assemblies and hydrating a reservoir in the device with a liquid. At least one of the electrode assemblies has a donor electrode and a donor reservoir for containing an ionic drug to be iontophoretically delivered. The donor reservoir is hydratable (e.g., having a liquid imbibing polymer) and upon hydration becomes applicable in drug transmitting relation with a body surface for iontophoretic delivery. The donor reservoir electrically communicates with a monitoring electrode at a location different from the donor electrode. The method further includes providing electrical communication by a monitoring circuitry to the donor electrode and the monitoring electrode for sensing impedance in the donor reservoir. The method further includes sensing impedance in the hydratable reservoir until an acceptable level has been achieved before enabling the device to be operational in therapeutic drug delivery current. The device can then be used on a body surface to allow current to flow through the body tissue under the body surface for electrotransport of the drug.

Providing conductivity measurement greatly improves the certainty that an iontophoretic drug delivery system will deliver the drug in the appropriate manner as designed. Without a reliable way of gauging the hydration of a hydratable gel, putting an iontophoretic drug delivery system that has just been hydrated on the skin always leaves a doubt that the system may not function to expectation. With improved reliability, hydratable matrix can become much better accepted in drug therapy as a means to provide systems that can be stored for long periods of time without fear of product failure because of drug degradation or electrical system breakdown due to corrosion. With the advent of fast hydrating reservoirs (such as liquid imbibing ester polymer disclosed herein), the convenience of being able to quickly hydrate a dry matrix and know quickly, perhaps instantly, that the reservoir is adequate for therapeutic ion migration will make dry matrix use not only viable, but a preferred mode of transdermal drug delivery.

Therefore, the present invention provides significant advance in iontophoretic drug delivery and great benefits to patients.

### BRIEF DESCRIPTION OF THE DRAWINGS

The present invention is illustrated by way of example in embodiments and not limitation in the figures of the accompanying drawings in which like references indicate similar elements. The figures are not shown to scale unless indicated otherwise in the content.

FIG. 1 illustrates a schematic, exploded view of a typical electrotransport device having impedance measurement circuitry of the present invention.

FIG. 2 illustrates shows a schematic representation of an embodiment of an electrotransport system having an impedance meter.

FIG. 3 shows another embodiment in which impedance of a donor reservoir can be measured.

FIG. 4 shows the in vitro flux of apomorphine from a TECOGEL® matrix after hydration.

FIG. 5 shows the impedance of a hydroxylethylcellulose-polyacrylic acid polymer matrix before hydration.

FIG. 6 shows the impedance of the hydroxylethylcellulose-polyacrylic acid polymer matrix of FIG. 5 during and after hydration.

FIG. 7 shows the impedance of TECOGEL® before hydration.

FIG. 8 shows the impedance of TECOGEL® during hydration.

FIG. 9 shows the impedance of a gel of PVP (poly vinyl pyrollidone) with propylene glycol under stepwise hydration.

### DETAILED DESCRIPTION

The present invention relates to an electrotransport system that includes an impedance meter or conductivity meter to determine the extent of hydration in a hydratable (liquid imbibing) reservoir in the system. Electrotransport drug delivery can be commenced after the impedance (or conductivity) has reached a predetermined condition or value.

In describing the present invention, the following terms will be employed, and are defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation.

“Biologically active agent” is to be construed in its broadest sense to mean any material that is intended to produce some biological, beneficial, therapeutic, or other intended effect, such as enhancing permeation or relief of pain. As used herein, the term “drug” refers to any material that is intended to produce some biological, beneficial, therapeutic, or other intended effect, such as relief of pain.

“Electrotransport” or “iontophoresis” refers to the delivery of pharmaceutically active agents (charged, uncharged, or mixtures thereof) through a body surface (such as skin, mucous membrane, ocular tissue) wherein the delivery is at least partially induced or aided by the application of an electric potential. The agent may be delivered by electromigration, electroporation, electroosmosis or any combination thereof. Electromigration involves the electrically induced transport of charged ions through a body surface by moving ions by means of a difference in electrical potential.

As used herein, the term “matrix” refers to a solid, or semi-solid substance, such as, for example, a polymeric material or a gel, that has spaces for a beneficial agent to populate and can hold a liquid for electrotransport. The matrix serves as a repository (as the structural or carrier material in a reservoir) in which the beneficial agent can be or is contained and may be porous. Unless specified, a matrix may or may not already have a beneficial agent included therein.

As used herein, the term “therapeutically effective” when applied to a drug or therapeutic agent refers to the amount of drug (therapeutic agent) or the rate of drug (therapeutic agent) administration needed to produce the desired therapeutic result.

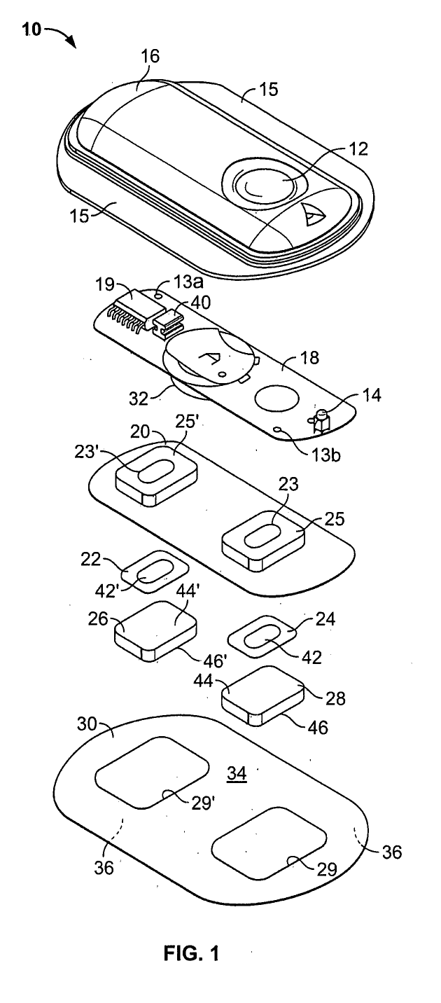

The resistance or conductivity meter is included in an electrotransport system, such as one similar to many of the prior disclosed electrotransport systems. For example, electrotransport systems such as those of U.S. Pat. Nos. 6,181,963; 6,317,629; and others can incorporate impedance meter or conductivity meter as described in the present invention. An iontophoretic system similar to that of U.S. Pat. No. 6,181,963 is shown in FIG. 1 and an impedance meter or conductivity meter can be provided and implemented with such a system. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable elastomer (e.g. ethylene vinyl acetate).

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′,29 in adhesive 30. Such a device can include a matrix of the ester polymer of the present invention in the system.

Printed circuit assembly 18 can contain a controller for controlling the operation of the device and even impedance sensing circuitry for monitoring the impedance of the donor reservoir. The device can also contain connectors with which an external circuit can be plugged and connected thereto. For example, an external ohmmeter or impedance meter can be connected.

The present invention is applicable to all systems that have a hydratable reservoir in which the level of hydration needs to be checked in situ (i.e., where the hydration reservoir is connected to the electrode that drives molecule migration in the reservoir). For example, impedance measurement circuitry can be implemented on an electrotransport device having a microprotrusion array and a reservoir disclosed in U.S. Patent Publication 20020016562 in a manner similar to the system of FIG. 1. With such a system, ions of larger molecular weights, e.g., in tens of thousands of Daltons, can be delivered. With such a system, no only small molecules, even large molecular weight biologics can be delivered.

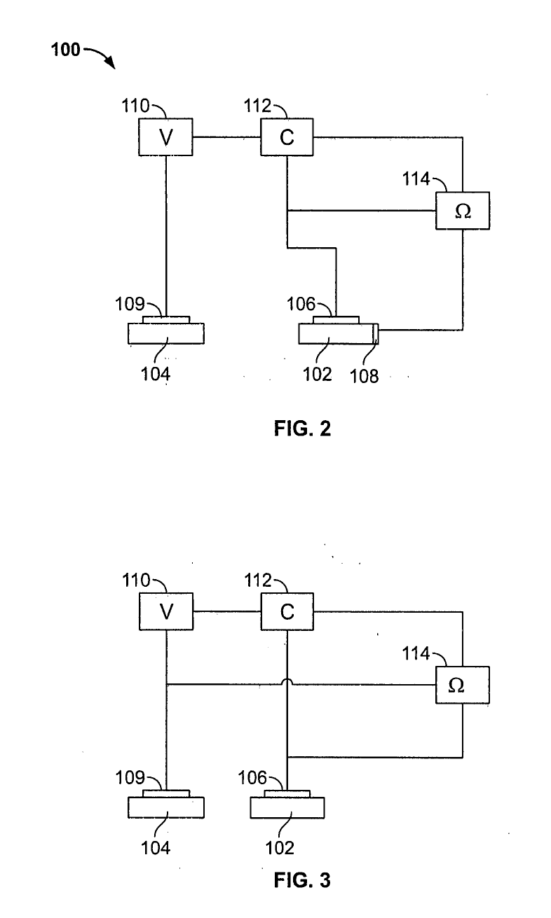

FIG. 2 shows a schematic representation of an embodiment of an electrotransport system having an impedance meter (ohmmeter) for determining the impedance to gauge the degree of hydration of a hydratable reservoir. In this figure the feature unrelated to the measurement of impedance is not shown so as to make the figure more easily understandable regarding impedance measurement. The electrotransport system 100 includes an ionic drug reservoir 102, a counter reservoir 104 that are to be placed on a body surface (not shown) for drug electrotransport. A donor electrode 106 contacts drug reservoir 102 to provide current for drug delivery. Counter electrode 109 contacts counter reservoir 104 for completing the electrical communication during electrotransport drug delivery to the body surface. Generally the donor electrode or a counter electrode is positioned centrally on a face of the corresponding reservoir so as to distribute current evenly over it. Voltage source 110 provides power for driving current flow. A Controller 112 that is operatively connected to the voltage source 110, donor electrode 106 and counter electrode 109 controls the operation of the electrotransport system, such as the duration of doses, turning the drug delivery system on or off based on various system conditions (e.g., out of range voltage or current flow), etc. An impedance meter 114 is connected to the donor electrode 106 and an auxiliary electrode 108 (which in turn is connected to the donor reservoir) to measure the impedance across the donor reservoir 102 between the donor electrode 106 and the auxiliary electrode 108. The impedance meter 114 is in electrical communication to the controller 1 12 to provide impedance information.

It is noted that a resistance meter, a impedance meter and a conductivity meter all amount to the same equivalent, in that the impedance or the inverse between two points are determined, i.e., whether it is determined in terms of impedance in ohms or conductivity in siemens (i.e., ohm⁻¹). Electrical impedance is a representation of how much an electrical component resists the flow of electrical current at a given voltage. It is denoted by the symbol Z and is measured in ohms. For something like a resistor under direct current the impedance will simply be the resistance. Impedance differs from simple resistance in that it takes into account possible phase offset under alternating current for components and circuits that have inductive or capacitive properties. For the purpose of estimating hydration we can generally consider impedance to be similar to resistance. Values of resistance and impedance can be determined and shown by devices such as ohmmeters and impedance meters.

The capability of measuring impedance, obtaining the real component (resistance) and the imaginary (i.e., reactance) component of the impedance of skin is within the ability of one skilled in the art. See, e.g., Kalia and Guy, “The Electrical Characteristics of Human Skin in Vivo”, Pharmaceutical Research, Vol. 12, No. 11, pp. 1605-1613, 1995, which is incorporated by reference herein. However, for the purpose of determining the level of hydration, using only the real component of impedance will normally suffice. Thus, in general, in the experimental measurement of impedance, either the resistance (real component) or the impedance with both real and imaginary components (reactance components) can be used. For example, when the capability to measure impedance including reactance is lacking, for the sake of simplicity, the hydration can be gauged by measuring only the real component (resistance).

The auxiliary electrode 108 can be positioned to the side face of the donor reservoir as represented by FIG. 2 or on the side of a face of the donor reservoir 102 as long as it provides consistent measurement of the impedance of the reservoir. In this way, the auxiliary electrode (or monitoring electrode) is outside of the space between the donor electrode and the body surface. With any particular electrode configuration, experimental analysis can be done by one skilled in the art to provide a correlation of the impedance with the degree of hydration in the reservoir.

It is preferred that none of the electrodes (i.e., the metallic part or similar part that has about zero impedance), including the donor electrode, counter electrode, or the monitoring electrode, directly contact the skin. Typically, each of such electrodes contact one the reservoirs such that current can flow through the reservoir to which the electrode is connected. This configuration enables the impedance of a reservoir to be measured.

It is noted that the impedance meter can be part of a body-surface-attaching unit, i.e., part of the portable electrotransport device that is attached to the body surface and carried around by the patient, or it can be a separate unit that is connectable and disconnectable to the portable electrotransport device. The circuits can be implemented on integrated circuit chips and installed either in a portable electrotransport device or placed in a separate unit that is connectable or disconnectable to the portable device. For example, either the portable electrotransport device or the impedance meter (or both) can have electrical receptors into which connectors from the other member of the electrotransport device/impedance meter pair can be physically inserted and frictionally fit or engage so that the connection can be frictionally maintained for impedance measurement without becoming disconnected. After the impedance measurement the connection is pulled apart on purpose to disengage the impedance meter. Such electrical receptors and connectors (e.g., prongs and sockets) are known in the art. Other connectors that can be used to engage the impedance meter with the portable electrotransport device can include clamps, clips, grips, and the like to provide disconnectable electrical communication. The designing of impedance measuring circuitry that can be implemented on a portable electrotransport unit is within the capability of one skilled in the art of such circuit design. For example, for a connectable impedance meter embodiment, the impedance meter can be plugged into a portable device to electrically communicate therewith for monitoring the impedance and forward signals relating to the impedance to the controller in the portable device. For a device that can be reused by replacing the hydratable reservoir(s) periodically, it is preferable that the impedance meter be on the portable device itself.

The electrodes can be made with typical materials known in the art. For example, the anode electrode can be made with silver, the cathode electrode can be made with silver chloride, and the auxiliary electrode can be made with silver, silver chloride, nonconsumable material such as carbon, other metallic materials such as platinum, gold, titanium, tungsten, stainless steel, gold-plated material, etc., known to one skilled in the art.

In an embodiment, at or after hydration but before the start of electrotransport drug delivery, i.e., before the full voltage and current for therapeutic drug delivery is switched on, a test current is sent or attempted to be sent through the donor reservoir 102 to determine the impedance of the reservoir between donor electrode 106 and auxiliary electrode 108. The magnitude of the test current is substantially less than what is necessary for driving therapeutic drug delivery, e.g., being less than 10% of the drug delivery current. Once the impedance of the donor reservoir has reached (i.e., fallen to) a desired range or level, the controller 112, receiving information by electrical communication from the impedance meter 114, will enable the therapeutic electrotransport drug delivery by making available the full voltage and current regime that is used for delivery of the drug for therapy. At this point, for a patient activated device, the user can initiate a therapeutic dose, for example, by pressing a button on the device. The system can also be designed for automatic drug delivery, such that the controller will start automatically a program of delivery, whereas before then the program cannot be started because the impedance has been too high. The device can have a display or alert (e.g., light or sound or both) to alert the user that the appropriate level of impedance has been reached or when the device is enabled to allow therapeutic drug flow by electrical current. Once the desired condition of impedance is reached, preferably no more hydration liquid (or no substantial amount) is added thereafter so the gel does not become too wet. For example, after the desired condition of impedance has been reached, no more than 20% more of the hydration liquid that has been added so far should be added.

Although it is possible to power the test current with the power source (battery) that drive therapeutic drug ions migration, the test current can be sent by the impedance meter from a power source that is different from the power source that drives the therapeutic drug ion migration.

The test current for sensing the resistance/impedance can be a direct current (DC) or an alternating current (AC). Using AC further provides an advantage that AC does not contribute to polarization of an electrode. A low test current and a low voltage are preferably used because according to Ohm's law a low voltage drives a low current for a particular impedance. For AC impedance (Z), the impedance (Z) is the sum of DC resistance (R), the inductive reactance (X_{L}), and the capacitive reactance (X_{L}), i.e., Z=R+X_{L}+S_{C}.

It has been found that the AC reactance components of the impedance are frequency dependent. However, as long as the level of hydration of a particular reservoir is calibrated against frequencies, the variation of impedance can be used to gauge the hydration of a reservoir. For example, if a consistent frequency is used (e.g., the consistent frequency can be selected to be a frequency within the range of 10 Hz to 100 KHz, or even outside this range), the impedance can be calibrated with reservoir samples of a particular type of hydratable polymer at various hydration levels. Regardless of AC or DC, the test current and the testing voltage for providing the test current can be much lower than (e.g., being less than 10%) those needed for therapeutic drug delivery. The frequency of the AC has an effect on the value of the impedance obtained in the measurement. A frequency can be chosen for convenience of measurement. As long as the same frequency is used in measuring an unknown sample and in a standard sample, one can readily gauge the extent of hydration in the unknown sample. Applicable frequencies can vary from a few Hz to hundreds of KHz, with a preference of 10 Hz to 10 KHz, preferably 500 Hz to 1000 Hz. Obviously, as long as the test current is useful in determining the impedance, the scope of the present invention is not limited by the magnitude of the test current.

FIG. 3 shows yet another embodiment in which impedance of the donor reservoir can be measured. In this embodiment, the impedance meter 114 is connected to the donor electrode 106 and to the counter electrode 109 such that before the start of the electrotransport, a test current is sent between the two electrodes to determine the overall impedance between the donor electrode and the counter electrode 109, including the resistance of the donor reservoir 102, the counter reservoir 104, and the skin resistance. A drug delivery current of a magnitude adequate for therapeutic drug delivery is commenced after the impedance of the whole system is determined to have reached (fallen to) a level or condition that is suitable for drug delivery after hydration. As described above, the test current is substantially smaller than the drug delivery current, e.g., less than 10% of the drug delivery current. A person skilled in art will understand that features that are applicable in the embodiment of FIG. 2 can similarly be applied in the embodiment of FIG. 3.

It is noted that in the drug delivery system, the impedance meter and the controller can be separate units, or they can be an integral unit that can perform both functions. In fact, any of the integral unit, the controller unit, and the impedance-monitoring unit can be an ASIC (application specific integrated circuit) or a design that incorporate programmable microprocessors or other discrete logic circuits and analog circuits. The designs of impedance measurement circuits, control circuits that can control voltage level and current level based on predetermined conditions such as changes in voltage, current, impedance, time, or other events are known to circuit designers skilled in the art. The controller unit and the controller can be both present in the drug delivery system that is attachable to the body surface (“patch”). Alternatively, the impedance monitoring unit can be a separate unit that is physically connectable and disconnectable to plug into the controller unit for electrical communication. In this way, the impedance monitoring unit can be reused repeatedly by a clinician for different body surface attachable patches.

The controller preferably controls the operation of the drug delivery system and directs the direction and magnitude of current flow through the various electrodes and their voltages such that the right levels of current and voltage are used for effective therapeutic ionic drug delivery by electrotransport, i.e., via a potential difference between the donor electrode and the counter electrode. Preferably the controller has circuitry that prevents a current flow to the body surface when the current or voltage during drug delivery is outside a predetermined range (based on safety reason) as can be determined by those skilled in the art. The controller can also control the drug delivery device to deliver the drug according a regime, for example, dose, time interval, etc. Preferably, an advantageous feature of the controller is that it has the circuitry, either by programmable logic, or hardwired circuit, that can switch on to enable the drug delivery current flow from the donor reservoir, through the body surface, such as that of the skin, to the counter reservoir. Further, it is preferred that the system has a monitoring circuitry that monitors the impedance even during drug delivery so that if the impedance goes outside a desirable range, e.g., as when the device becomes detached from the body surface, the controller will switch off the current flow to the donor reservoir. Drug delivery current flow can be reinitiated when the impedance returns to the desirable range. Preferably, the controller has circuitry that prevents the current flow to the body surface when impedance across the donor reservoir is above a predetermined level.

The desirable impedance across a donor reservoir is somewhat dependent on the particular drug being delivered because certain drugs only require a relatively small current to deliver the therapeutic dose. However, typically an impedance that is above about 1 to 10 Kohms would not allow adequate drug flow for most ionic drugs with a reasonable voltage in a battery operated skin patch electrotransport device due to compliance voltage max out. Generally the desirable impedance across the donor reservoir is about below 500 ohms, preferably about 100 ohms to 500 ohms, more preferably about 50 ohms to 200 ohms.

In an instance in which impedance is measured between the donor electrode and the counter electrode, the body surface tissue (e.g., skin tissue) impedance is also included in the measure. Generally, the impedance of the body surface tissue, e.g., skin, can vary depending on factors such as the amount of hydration of the tissue, especially the stratum corneum, and whether the tissue has experienced electrotranport (since the impedance of the skin tends to fall with electrotransport). The impedance of the skin, depending on the frequency of the current used for measurement, is generally is about a few Kohms to hundreds of Kohms. See e.g., Kalia and Guy, “The Electrical Characteristics of Human Skin in Vivo”, Pharmaceutical Research, Vol. 12, No. 11, pp. 1605-1613, 1995. Thus, the impedance of the skin between the reservoirs is the combination of the impedance of the tissue and the reservoirs. However, the scope of the present invention is not dependent on the specific values of the body tissue or the reservoir, as long they are within the range that can be measured by impedance measuring equipment and methods.

A reservoir, e.g., a drug donor reservoir or a counter ion reservoir, can be made with a hydratable material and be hydrated at the time of need for the electrotransport drug delivery. The reservoir can be made with liquid imbibing material known in the art. For example, the reservoir can be made of a dried hydrogel or have a support material which can hold a liquid solution or gel material. A hydrogel can be a polyethylene oxide polymer that is cross-linked. Suitable hydrophilic polymers for hydrogels include polyvinylpyrrolidones, polyvinyl alcohol, polyethylene oxides such as POLYOX® manufactured by Union Carbide Corp., CARBOPOL® manufactured by BF Goodrich of Akron, Ohio; blends of polyoxyethylene or polyethylene glycols with polyacrylic acid such as POLYOX blended with CARBOPOL, polyacrylamide, KLUCEL®, cross-linked dextran such as SEPHADEX (Pharmacia Fine Chemicals, AB, Uppsala, Sweden), WATER LOCK® (Grain Processing Corp., Muscatine, Iowa) which is a starch-graft-poly(sodium acrylate-co-acrylamide) polymer, cellulose derivatives such as hydroxyethyl cellulose, hydroxypropylmethylcellulose, low-substituted hydroxypropylcellulose, and cross-linked Na-carboxymethylcellulose such as AC-DI-SOL (FMC Corp., Philadelphia, Pa.), polyhydroxyethyl methacrylate (National Patent Development Corp.), natural gums, chitosan, pectin, starch, guar gum, locust bean gum, and the like, along with blends thereof. The support material can be, e.g., a hydrophilic foam such as a polyurethane foam, a nonwoven porous polyester, a fibrous or cloth material, etc. Hydrophilic thickener can be present in the support material, e.g., high molecular weight polyethylene oxide (PEO), high molecular weight polyvinyl alcohol (PVA), poly-N-vinyl pyrrolidone (PVP), or other substituted pyrrolidones, polyacrylamide (PAAm), poly-N-isopropyl acrylamide (NIPPAm), polyhydroxyethyl methacrylate (PHEMA), or hydrophilic substituted HEMAs, polysaccharides such as agarose, hydroxyethyl cellulose (HEC), hydroxypropylmethyl cellulose (HPMC), hydroxypropyl cellulose (HPC), carboxymethyl cellulose (CMC), dextrans, modified starches, modified collagens, xanthan gum, guar gum, modified natural gums, partially neutralized polyelectrolytes such as polyacrylic acid, polyimides, and alginates. In some circumstances, copolymer mixtures of the above may also be suitable. The polymer is selected to provide the desired viscous property in the hydrated matrix for handling and electrotransport, as would be known to one skilled in the art.

Another kind of polymer that can be used for forming hydratable water-imbibing reservoirs is a polymeric ester that has acid groups that are not esterified so that the carboxyl groups are free to associate with cationic drugs. The polymeric ester is a polymer having a monomer component that is an acid polymer (e.g., polyacrylic acid (PAA)) and a monomer component that is a hydroxyl polymer. The ester is formed by a reaction between the free carboxyl groups of an acid polymer with the hydroxyl groups of a second polymer (an hydroxyl polymer) to form a covalent ester cross-link. It is preferred that the hydroxyl polymer has multiple hydroxyl groups and the acid polymer has multiple carboxyl groups for cross-linking. A class of substance useful as the hydroxyl polymer is hydroxyalkyl polymer. Such a hydroxyalkyl polymer will have hydroxyl group —OH connected to another group through an alkyl linkage in the polymer, i.e., having a —OH connected via single bonded hydrocarbon link (e.g., —CH₂—) to other groups in the polymer. Preferably, the —OH is connected via a single bonded hydrocarbon link to an oxygen in an ether linkage. Preferably, the single bonded hydrocarbon link is one to three carbons long. More preferably the single bonded hydrocarbon link is one to two carbons long, e.g., —CH₂—CH₂— as in a hydroxyethyl group. Further, it is preferred that there are ether linkages connecting repeated moieties in the polymer, as in for example, polyethylene glycol polymer, alkylene oxide (e.g., ethylene oxide, propylene oxide) polymer, and carbohydrate like structures.

A useful type of hydroxyalkyl polymer includes carbohydrates such as polysaccharides and their derivatives. Such carbohydrates and their derivatives contain polymerized saccharose ring structures. Carbohydrate derivatives are useful as long as they have hydroxyl groups, especially primary or secondary hydroxyl group, that can form ester with an acid polymer. Preferably the hydroxyl polymer is cellulosic as a cellulose derivative. Preferred cellulosic hydroxyl polymers include hydroxyalkyl cellulose such as hydroxyethyl cellulose, hydroxypropyl cellulose, hydroxypropyl methyl cellulose, ethyl hydroxyethyl cellulose, and the like. One of the advantages afforded by the polysaccharides and especially cellulosic hydroxyl polymers is their liquid absorbing capacity, particularly in absorbing aqueous solutions. Another advantage is that they can form films with good mechanical properties such as flexibility and toughness. Other preferred hydroxyl polymers include starch and starch derivatives, maltodextrin, chitosan, and natural gums such as locust bean gum, guar gum, carrageenin, agar, and carob gum, and their derivatives.

Another class of hydroxyl polymers is linear polymers without ring structures, preferably with hydroxyl groups at both ends of the polymer. For example, hydroxyl polymers with blocks of ethylene oxide units are useful. Examples of such ethylene oxide containing hydroxyl polymers include polyvinyl alcohol-polyethylene glycol graft copolymer and ethylene oxide-propylene oxide-ethylene oxide triblock copolymers.

Polyvinyl alcohol-polyethylene glycol graft copolymer is also a preferred hydroxyl polymer for forming the ester. The polyethylene glycol chains of this polymer have primary —OHs at the ends thus providing the needed reactivity and additionally the graft copolymer inherently has good film forming and tensile properties.

The acid polymer for forming the ester is a polymer having repeating units with acidic carboxyl groups such that when these carboxyl groups form a covalent bond and cross-link with the hydroxyl polymer, they result in a cross-linked ester and thus achieve a liquid-imbibing yet insoluble structure. Under appropriate condition of liquid incorporation, the matrix can have a gel-like consistency with homogeneous physical property throughout the matrix. Examples of such acid polymers include polyacrylic acid, polymethacrylic acid, polyethylacrylic acid, copolymers of methyacrylic acids such as ethyl acrylate/methacrylic acid copolymers, cellulose acetate phthalate, hydroxypropyl methylcellulose acetate succinate, hydroxypropyl methylcellulose phthalate, polyvinyl acetate phthalate, and cellulose acetate trimellitate, alginic acid, and pectic acid, gelatin, casein, arachin, glycinin, and zein, some of which are polypeptides and proteins. Such acid polymers can have pendant groups substituted and can be homopolymers or copolymers, as long as they have multiple carboxyl groups reactive to —OH groups in the hydroxyl polymer to form an ester.

To react with the hydroxyl polymer, especially preferred is polyacrylic acid. The polyacrylic acid can either be cross-linked or noncross-linked. However, if the polyacrylic acid is cross-linked, the amount of cross-linking is sufficiently low that the polyacrylic acid can absorb a large amount of water. Useful polyacrylic acids commercially available include CARBOPOL® polyacrylic acids (which are presently, at 2006 A.D., available from Noveon, Inc., 9911 Brecksville Road, Cleveland, Ohio), such as CARBOPOL 907 (which is not cross-linked), CARBOPOL 980 (which is cross-linked), CARBOPOL 940 and CARBOPOL 2984, and the like. The more preferred polyacrylic acids are either soluble in water or can absorb a large amount of water (e.g., 100 times by weight, preferably more than 500 times by weight, more preferably more than 1000 times by weight) at about neutral pH to form a homogenous material. The viscosity of preferred polyacrylic acid when dissolved at a concentration of 0.5 weight percent in pH 7.5 buffer is preferably in the range of about 1,000 to 80,000 centipoises, preferably 40,000 to 60,000 centipoises as measured by a Brookfield viscometer at 20 revolutions per minute. For cross-linked polyacrylic acid, preferably, the molecular weight is such that if the cross-linked polyacrylic acid were without cross-linker (i.e., made from the same ingredients but without using cross-linker), the weight average molecular weights are about 200,000 to 1,000,000, preferably 400,000 to 600,000 as measured by gel permeation chromatography using linear polyacrylic acid as reference. Therefore, in the polyacrylic acid, there are many —COOH groups that can react with the hydroxyl polymer.

The ratios of hydroxyl polymer to carboxyl polymer can be determined experimentally to identify practical ranges. In general, using a lower amount of acid polymer (e.g., using a lower concentration of polyacrylic acid) will yield an ester polymer film that when hydrated is jelly-like with low mechanical integrity. Generally, to form reservoirs for iontophoretic drug delivery, the ester polymer in film form is a convenient structure. Such a film can be cut into small sizes to be placed in an iontophoretic device. A larger amount of the acid polymer in the reaction (e.g., using a higher concentration of PAA) would result in an ester polymer film that in the dry state is too brittle to handle. For example, using the same wt % solutions of PAA and HEC, with PAA solution ranging about 10 to 30 vol % in the mixture is suitable, with about 15 to 25 vol % being preferred, to avoid these extremes in mechanical properties. In view of the present disclosure, one skilled in the art will know other variations of wt % solutions of each reactant and the mixture vol % to use for the two solutions.

Synthesis of the polymeric ester can be done through a condensation reaction potentiated by heat and vacuum between the free carboxyl groups of the carboxyl polymers and the free hydroxyl of hydroxyl polymers to form a covalent ester cross-link. For example, the reaction can be done in a vacuum oven with a vacuum of 600-760 mm Hg and a temperature in the range of 40-80° C. The cross-link causes the resulting polymeric ester to become insoluble in water (thereby permitting less polymer residue being left on the body surface, e.g., skin, when the delivery system is removed therefrom). After the polymer is formed it can be dried and then placed in a drug solution to incorporate the drug.

The polymer with the drug loaded thereon can be dehydrated to form a dry hydratable material that can imbibe liquid to result in a reservoir for electrotransport. Prior to electrotransport, treating the dry drug-containing polymer with a suitable solvent frees the ionic drug to be moved by the application of an electrical potential. The hydration step allows the bound drug molecules to dissociate from the reservoir (e.g., carboxyl groups of the ester gel) and can be any aqueous or polar organic solvent that will allow the drug ions to flow under the influence of an electric field. Hydrating the ester polymer with a solvent or solvent mixture requires the use of a polar liquid capable of solvating the drug ion and preserving it in an ionic state for electrotransport delivery. Solvents used for this include organic solvents, inorganic solvents, solution of various solvents, buffers, and the like that one skilled in the art will know related to the drug. Such solvents include, but not limited to: water, ethanol, ethanol:water blends (especially useful at 70:30 to 30:70 ratios), methanol, methanol:water blends, glycerin, glycerin:water blends, propylene glycol, propylene glycol:water blends, dimethyl sulfoxide, dimethyl sulfoxide:water blends, glycerol oleate solution, low molecular weight polyethylene glycol (PEG, e.g., PEG 400), PEG:water blends, PEG 660 12-hydroxy stearate (note: paste at room temp but liquid at skin temp), and combinations thereof.

Hydration of a hydratable reservoir of the present invention can be done using, for example, a pipette or syringe type of device or other devices that provide a controlled volume of hydrating liquid. From the start to the finish of the hydration process, the impedance of the reservoir material can be monitored to determine the progress of hydration. When adequate hydration is determined to have occurred, the device can then be safely used for drug delivery. The acceptable level of hydration is achieved when a precipitous drop of impedance from a very high value to a stable low value is shown, indicating that ions can migrate through the reservoir readily. Typically, before hydration, the impedance across the dry, unhydrated donor electrode is almost infinite. As the hydratable polymer imbibes liquid, the impedance falls in a fashion that is nonlinear but looks exponential. For a fast hydrating hydratable polymeric matrix, such as one made of a polyacrylic acid—hydroxyethyl cellulose ester, an acceptable impedance that indicates adequate hydration for electrotransport can be achieved in a matter of minutes, even as little as one minute, or less.

For convenience, a kit including a portable electrotransport device with dehydrated reservoir and a hydrating liquid source can be provided, so that the exact amount of hydrating liquid has been premeasured for the reservoir to be hydrated. For example, the hydrating liquid can be in a container with a tip for depositing the liquid in the hydratable reservoir. The portable electrotransport device can include the impedance meter or is connectable to a separate impedance meter as described above.

Various biologically active agents or drugs may be incorporated in the reservoir matrix of the present invention for use in treating individual in need of treatment by such drugs. The biologically active agents or drugs can be incorporated by imbibition and drying. The drug containing matrix can then be hydrated before drug delivery. Such biologically active agents or drugs include cationic drugs that are known to those skilled in the art. Agent or drugs that can be incorporated into the matrix include, for example, interferons, alfentanyl, amphotericin B, angiopeptin, baclofen, beclomethasone, betamethasone, bisphosphonates, bromocriptine, buserelin, buspirone, calcitonin, ciclopirox, olamine, copper, desmopressin, diltiazem, dobutamine, dopamine agonists, dopamine agonists, doxazosin, droperidol, enalapril, enalaprilat, fentanyl and its analogs and salts thereof (such as alfentanil, carfentanil, lofentanil, remifentanil, sufentanil, trefentanil), encainide, G-CSF, GM-CSF, M-CSF, GHRF, GHRH, gonadorelin, goserelin, granisetron, haloperidol, hydrocortisone, indomethacin, insulin, insulinotropin, interleukins, isosorbide dinitrate, leuprolide, LHRH, lidocaine, lisinopril, LMW heparin, melatonin, methotrexate, metoclopramide, miconazole, midazolam, nafarelin, nicardipine, NMDA antagonists, octrebtide, ondansetron, oxybutynin, PGE 1, piroxicam, pramipexole, prazosin, prednisolone, scopolamine, seglitide, sufentanil, terbutaline, testosterone, tetracaine, tropisetron, vapreotide, vasopressin, verapamil, warfarin, zacopride, zinc, and zotasetron, individually or in combination.

The hydratable matrix material is useful for incorporating agents or drugs such as peptides, polypeptides and other macromolecules typically having a molecular weight of at least about 300 daltons, and typically a molecular weight in the range of about 300 to 40,000 daltons. Specific examples of peptides and proteins in this size range include, without limitation, LHRH, LHRH analogs such as buserelin, gonadorelin, nafarelin and leuprolide, GHRH, insulin, heparin, calcitonin, endorphin, TRH, NT-36 (chemical name: N-[[(s)-4-oxo-2-azetidinyl carbonyl]-L-histidyl-L-prolinamide), liprecin, pituitary hormones (e.g., HGH, HMG, HCG, desmopressin acetate, etc.,), follicle luteoids, aANF, growth hormone releasing factor (GHRF), βMSH, TGF-β, somatostatin, atrial natriuretic peptide, bradykinin, somatotropin, platelet-derived growth factor, asparaginase, bleomycin sulfate, chymopapain, cholecystokinin, chorionic gonadotropin, corticotropin (ACTH), epidermal growth factor, erythropoietin, epoprostenol (platelet aggregation inhibitor), follicle stimulating hormone, glucagon, hirulogs, hyaluronidase, interferons, insulin-like growth factors, interleukins, menotropins (urofollitropin (FSH) and LH), oxytocin, streptokinase, tissue plasminogen activator, urokinase, vasopressin, ACTH analogs, ANP, ANP clearance inhibitors, angiotensin II antagonists, antidiuretic hormone agonists, antidiuretic hormone antagonists, bradykinin antagonists, CD4, ceredase, CSF's, enkephalins, FAB fragments, IgE peptide suppressors, IGF-1, neuropeptide Y, neurotrophic factors, opiate peptides, parathyroid hormone and agonists, parathyroid hormone antagonists, prostaglandin antagonists, pentigetide, protein C, protein S, ramoplanin, renin inhibitors, thymosin alpha-1, thrombolytics, TNF, vaccines, vasopressin antagonist analogs, alpha-1 anti-trypsin (recombinant).

Other drugs that can be incorporated in the reservoir matrix include diphenylmethane derivatives with antihistaminic activity such as cyclizine, chlorcyclizine, bromodiphenhydramine, diphenylpyraline, diphenhydramine, chlorcyclizine, medrilamine, phenyltoloxamine clemastine; pyridine derivatives with antihistaminic activity such as chlorpheniramine, brompheniramine, pheniramine, mepyramine, tripelennamine, chloropyramine, thenyidiamine, methapyrilene; diphenylmethane derivatives with anticholinergic activity such as adiphenine, piperidolate, benztropine, orphenadrine, chlorphenoxamine, lachesine, poldine, pipenzolate, clidinium, benzilonium, ambutonium; anticholinergic agents such as oxybutynin, oxyphenonium, tricyclamol, dicyclomine, glycopyrronium, penthienate; antidepressant drugs such as fluoxetine, iprindole, imipramine, clomipramine, desipramine, trimipramine, amitriptylline, nortriptylline, noxiptiline, butriptiline, doxepin, dothiepin, iprindole, protryptiline, melitracene, dimetacrine, opipramol, paroxetine, sertraline, citalopram; tranquillizers such as promazine, chlorpromazine, chlorproethazine, methoxypromazine, methpromazine, promethazine, dimethothiazine, methiomeprazine, trimeprazine, methiotrimeprazine, diethazine, thioridazine, perazine, trifluoperazine, thioperazine, thiethylperazine, perphenazine, fluphenarine thiopropazate, thiothixene, chlorprothixene; antipsychotics such as pimozide, thiopropazate, flupenthixol, clopenthixol, trifluoperazine, olanzapine; anorexics such as fenfluramine and chlorphentermine; analgesics such as methadone and dextropropoxyphene; local anaesthetics such as tetracaine, stadacaine, cinchocaine, lidocaine; antihypertensives such as propranolol, oxprenolol, acebutolol, sotalol, metoprolol; antiarrhythmic and antianginals such as amiodarone, dilthiazem and verapamil; antiestrogen such as tamoxifen; and antiosteoporotic agents such as raloxifen. Cationic drugs that are mentioned in U.S. Pat. No. 6,181,963 can also be used and are incorporated by reference herein.

Traditionally most drugs have been small molecules, e.g., chemicals, antibiotics, etc., many of which are manufactured by chemical synthesis. Nowadays, biologics are becoming important. Biologics are generally large complex molecules (typically proteins) that are derived or manufactured from living cells. Examples of biologics include vaccines, blood products, cytokines, monoclonal antibodies, hormones, and the like. Biologics are especially prone to degradation. Certain agents or drugs, especially biologics, proteins, polypeptides, polynucleotides, and the like, may degrade in solution rapidly. In solution, some may have less than 90% recovery at room temperature within one week, or even less. Some may be unstable to the extent that recovery from solution is 80% or less in 3 weeks, 2 weeks, or even 1 week. Thus, many biologically active agents need to be stored in dry state. Such biologically active agents or drugs will benefit from employing the hydratable matrix for dry storage before hydration.

The drug reservoir having hydratable polymer can be placed in an electrotransport device such as one shown in FIG. 1 with the impedance measuring features of FIG. 2 or FIG. 3 or their variations, prior to hydration. When placed in the device, the drug reservoir will be in contact with current distribution parts such as silver or silver chloride electrodes and can contact body surface after hydration for drug delivery.

### EXAMPLES

Below are examples of specific embodiments for carrying out the present invention. The examples are offered for illustrative purposes only, and are not intended to limit the scope of the present invention in any way. In the following examples all percentages are by weight unless noted otherwise. In the following examples, the impedance measurements were done using a 1V AC test current at 962 HZ unless otherwise stated differently.

### Example 1

### In Vitro Flux of Apomorphine in TECOGEL®

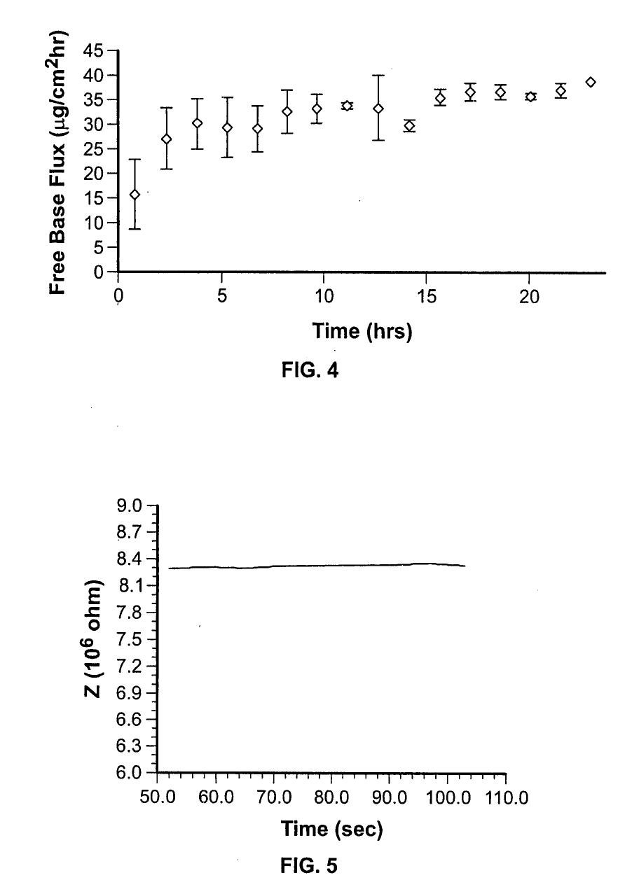

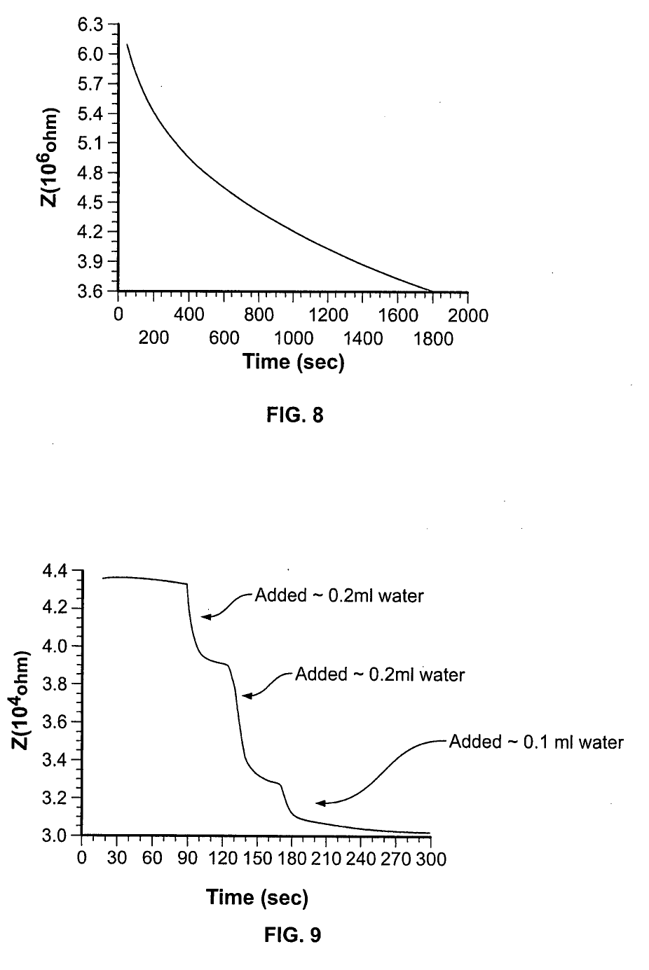

FIG. 4 shows the in vitro flux of apomorphine from a TECOGEL® (engineered polyurethane, Noveon, Incorporated, Cleveland, Ohio) matrix after hydration. The diamond symbols show the average data points at particular time intervals and the vertical lines show the standard deviation. Measurement of impedance during hydration of TECOGEL® is reported in FIG. 8 and described in Example 3 below. The flux of apomorphine free base is plotted as a function of electrotransport time (in hrs). The unit for the flux is μg/cm² hr. The plot shows a rise to steady state time of about 3 hours and thereafter a steady state flux of about 32 μg/cm² until termination of readings at about 24 hours. This shows that a hydrated matrix can support drug flux. It is noted that the TECOGEL® was used to illustrate that hydration can be done and the level of hydration measured. The exact type of polyurethane gel used is not critical as long a gel can be formed that can allow drug ions to migrate under an electrical potential.

The following methodology was used for the in vitro flux experiments for illustrative purposes:

Custom-built horizontal diffusion cells made in-house from DELRIN® polymeric material were used for the in vitro skin flux experiments and heat separated human epidermis was used. A consumable Ag electrode with the same polarity as the drug was adhered to one end of a DELRIN® material diffusion cell that functioned as the donor cell. The counter electrode made of AgCl was adhered at the opposite end. These electrodes were connected to a current generator (Maccor) that applied a direct current across the cell. The Maccor unit was capable of applying a voltage up to 20V to maintain constant iontophoretic current.

In a typical experiment, the heat separated human epidermis was punched out into suitable circle 24 mm ( 15/16 in) diameter and refrigerated just prior to use. The skin was placed on a screen 24 mm ( 15/16 in) that fitted into the midsection of the DELRIN® housing assembly. Underneath the screen was a small reservoir that was 13 mm (½ in) in diameter, 1.6 mm ( 1/16 in) deep and could hold approximately 250 μl of receptor solution. The stratum corneum side of the skin was placed facing the drug containing hydrogel and the epidermis side faced the receptor reservoir. The receptor solution (saline, phosphate or other buffered solutions compatible with the drug) was continuously pumped through the reservoir via polymer tubing (Upchurch Scientific) connected to the end of a syringe/pump assembly. The drug containing polymer layer was placed between the donor electrode and heat separated epidermis. A custom-built DELRIN® spacer was used to encase the drug layer such that when the entire assembly was assembled together, the drug-containing polymer was not pressed too hard against the skin as to puncture it. Double-sided sticky tape was used to create a seal between all the DELRIN® parts and to ensure there were no leaks during the experiment. The entire assembly was placed between two heating blocks that are set at 37° C. to replicate skin temperature.

A Hanson Research MICROETTE™ collection system, interfaced to the experimental set up, collected the drug containing receptor solution from the reservoir underneath the skin directly into HPLC vials. The collection system was programmed to collect samples at specified time intervals depending on the length of the flux experiment, for example, at every hour for 24 hours. The Hanson system collected samples to be analyzed by an HPLC to determine delivery efficiency of the drug in the formulation.

A 1/10 diluted Delbeccos phosphate buffered saline (DPBS) receptor solution was used as the receiver fluid because it had the same concentration as the endogenous fluid. The DPBS was pumped into the receptor solution reservoir at 1 ml/hr. The drug solution (apomorphine in water containing antioxidants) was introduced into the TECOGEL® matrix by imbibing. The drug-containing TECOGEL® (Neveon) polymeric material was then placed in the donor compartment next to the Ag electrode. A hydration step was done prior to electrotransport—a drop of water was added to hydrate the film prior to turning on the current.

### Example 2

### Hydration of HEC-CARBOPOL Film

An ester polymer formed by the condensation reaction between hydroxyethyl cellulose (hydroxyl class) NATROSOL 250 and CARBOPOL polyacrylic acid (carboxyl class) CARBOPOL 980 was used for the case study. Impedance during hydration was measured. The following method was used.

A custom made conductivity test cell made of DELRIN® (DuPont) with stainless steel disc electrodes was used for impedance measurements. The unit had a micrometer attached to spring loaded electrodes to measure the thickness of the sample. Stainless steel screws at both the ends served as the connecting leads. The effect of hydration on impedance was tested by placing conductivity cell with the polymer film (matrix) in a vertical fashion and introducing water via opening of the electrodes while keeping the polymer film intact on one of the electrodes.

One of the disc electrode was connected to the working electrode (equivalent to donor electrode 106 in FIG. 2) and the other disc electrode was connected to the monitoring lead (or auxiliary electrode) (equivalent to monitoring electrode 108 in FIG. 2) of a CH Instrument electrochemical work station to measure impedance. The reference electrode lead of the CH instrument was also connected to the monitoring electrode. Impedance measurements were carried out by application of a very small AC voltage (1 V) at a frequency of 962 Hz. The run time varied between samples (110-250 sec) and data were measured every 10 sec.

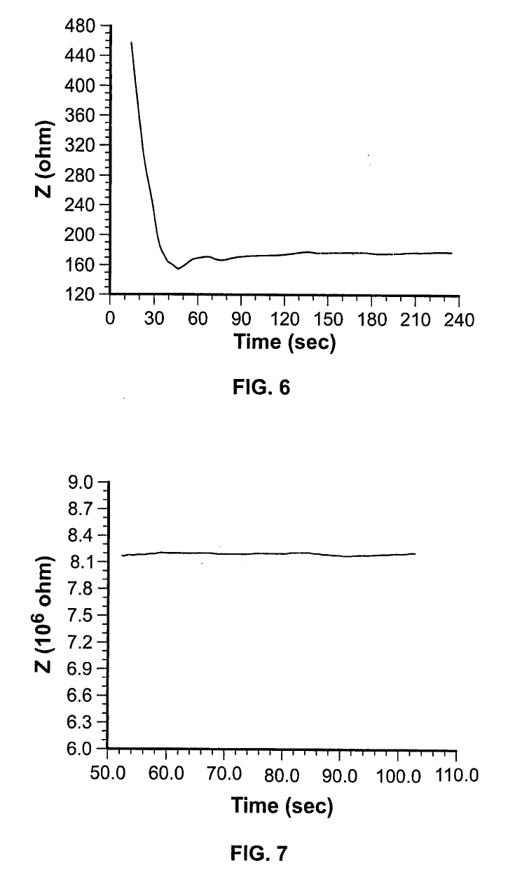

FIG. 5 shows the impedance measurements. Impedance was measured by CH Instruments meter Model 860A. The impedance (Z) versus time (T) plot in FIG. 5 shows an impedance value of about 8.3×10⁶ ohms before hydration over a period of about 2 minutes. In the figure, the impedance measurement is shown in units of 10⁶ ohms. The impedance of that material upon hydration is shown in FIG. 6, which shows impedance in units of ohms versus time in seconds. (The abscissa shows time in seconds.) FIG. 6 shows that upon hydration using 10 μl of water on a hydratable reservoir of typical size (about 1.3 cm², thickness of about 0.2 cm after hydration) for an iontophoretic system, there was a rapid drop in the impedance within the first 50 seconds. Within 50 seconds the impedance fell to below 160 ohms. The impedance subsequently stabilized to a value of around 160 ohms at about a minute after depositing the water on the matrix. This illustrates that a hydratable reservoir can be quickly hydrated.

### Example 3

### Hydration of TECOGEL® Film

An engineered polyurethane based TECOGEL® was used as the sample and Z-T plot on FIG. 7 shows the impedance of the TECOGEL® in the prior-to-hydration state. The impedance is shown in units of 10⁶ ohms. The abscissa shows time in seconds. Hydration was done on a hydratable reservoir of typical size (about 1.3 cm², thickness of about 0.2 cm after hydration) for an iontophoretic system. Impedance measurement can be done similarly with other electrotransport systems as long as hydratable reservoirs are included. The gel showed a baseline value of about 8.2×10⁶ ohms over a period of about 2 minutes before hydration. Upon hydration, the impedance as shown in the plot on FIG. 8 shows a slow decay of the impedance to about 3.6×10⁶ ohms in about 1800 seconds, at which time the impedance was still falling. In FIG. 8, the impedance is also shown in units of 10⁶ ohms.

The change in impedance upon hydration for the different materials shows the sensitivity of the method to identify hydration kinetics based on material properties. The HEC-CARBOPOL ester polymer due to its hydrophillicity hydrated quickly while the polyurethane based TECOGEL® being hydrophobic was resistant to hydration as shown in the impedance values.

### Example 4

### Hydration of PVP (poly vinyl pyrollidone) Film

A polymer of PVP (poly vinyl pyrollidone) containing added propylene glycol as an excipient was hydrated with water and the impedance measured. The impedance during hydration is shown in FIG. 9, which shows impedance on the ordinate in units of 10⁴ ohms. The abscissa shows time in seconds. Hydration was done on a hydratable reservoir of typical size for an iontophoretic system (about 1.3 cm²×0.2 cm after hydration). The materials showed a baseline impedance of 4.4×10⁴ ohms before hydration. The impedance fell after the addition of 0.2 ml water to the polymer film. The plot shows a systematic decrease as additional amounts of water were added. The drop in impedance was higher in the instances with the addition of 0.2 ml of water in the first part of hydration compared to that with 0.1 ml addition in the later part. This plot shows the sensitivity of the technique of hydration determination by impedance measurement.

The above examples are for illustrating that the extent of hydration can be determined using the systems and methods of the present invention. The specific reservoir materials and hydration processes are for illustrating the determination of hydration. Regardless of the speed of hydration, whether it is in seconds of over a period of many minutes, the present invention can be used to estimate the extent of hydration to provide a means of determining whether the hydratable reservoir is ready for electrotransport drug delivery.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention. All patent and patent application document references cited in the present disclosure are hereby incorporated by reference in their entireties herein.

What is claimed is:

1. An iontophoretic agent delivery device comprising a pair of electrode assemblies, at least one of said electrode assemblies having a donor electrode and a donor reservoir for containing an ionic drug to be iontophoretically delivered, said donor reservoir being hydratable and upon hydration becoming applicable in drug transmitting relation with a body surface for iontophoretic delivery, the device having a monitoring electrode capable of electrical communication with a monitoring circuitry for sensing impedance in the reservoir.

2. The device of claim 1 wherein the donor reservoir before dehydration is dry and a monitoring circuitry is electrically connected to the monitoring electrode and to the donor electrode or electrically connected to connectors connectable to the monitoring electrode and to the donor electrode to sense the impedance between the monitoring electrode and to the donor electrode, the monitoring circuitry to monitor the impedance by sending through the donor reservoir a testing current that is ineffective for driving the ionic drug for drug delivery.

3. The device of claim 2 comprising circuitry that controls iontophoretic drug delivery, wherein the control circuitry in electrically communication with the monitoring circuitry so that the control circuitry permits drug delivery current flow from the donor electrode to the body surface when impedance across the donor reservoir is sensed to have reached a predetermined condition.

4. The device of claim 2 comprising control circuitry that controls iontophoretic drug delivery, wherein the control circuitry is in electrically communication with the monitoring circuitry, the control circuitry controlling current flow from donor electrode such that drug delivery current flow from the donor electrode to the body surface cannot be turned on until impedance between the donor electrode and the monitoring electrode has been sensed by the monitoring circuitry to have reached a predetermined condition; wherein the monitoring circuitry uses a test current to sense the impedance.

5. The device of claim 2 comprising a counter electrode and a counter reservoir for completing path for current flow from the donor reservoir through the body surface, wherein the monitoring electrode is different from the counter electrode.

6. The device of claim 2 comprising a counter electrode and a counter reservoir for completing path for current flow from the donor reservoir through the body surface, wherein the monitoring electrode is different from the counter electrode and the monitoring electrode is in contact with the donor reservoir and is positioned outside space between the donor electrode and the body surface.

7. The device of claim 2 comprising a counter electrode and a counter reservoir for completing path for current flow from the donor reservoir through the body surface, wherein the monitoring electrode is the same as the counter electrode.

8. The device of claim 2 wherein the body surface is the surface of the skin and the device comprising a counter electrode and a counter reservoir for completing path for current flow from the donor reservoir through the skin, wherein the monitoring electrode is the same as the counter electrode and the device has control circuitry that switches on drug delivery current flow when the monitory circuitry determines that impedance from the donor electrode to the counter electrode including the skin impedance has reached a predetermined condition.

9. The device of claim 2 further comprising a circuitry transmitting an electrical signal from the monitoring circuitry to trigger action of another circuitry depending on the impedance sensed.

10. The device of claim 2 further comprising a circuitry transmitting an electrical signal to trigger action of a light or sound alert depending on the impedance sensed.

11. The device of claim 3 wherein the monitoring circuitry is connectable and disconnectable with the controller circuitry by friction fit connectors.

12. The device of claim 3 wherein the donor reservoir comprises a liquid imbibing ester polymer and a cationic drug.

13. The device of claim 13 wherein the donor reservoir comprises a liquid imbibing ester polymer and a cationic drug, the ester polymer having nonesterified carboxyl groups for noncovalently associating with the cationic drug.

14. The device of claim 3 wherein the donor reservoir comprises a liquid imbibing ester polymer and a cationic drug, wherein the ester polymer has monomeric component that is an hydroxyalkyl polymer and monomeric component that is an acid polymer, the acid polymer being one of polyacrylic acid polymer and polymethacrylic acid polymer.

15. An iontophoretic agent delivery device comprising:
a. donor electrode assembly having a donor electrode and a donor reservoir for containing an ionic drug to be iontophoretically delivered, said donor reservoir being hydratable and upon hydration becoming applicable on a body surface for iontophoretic delivery;
b. counter electrode assembly having a counter electrode and a counter reservoir for contacting the body surface to complete path for current flow;
c. monitoring electrode contacting the donor reservoir to determine the impedance across the donor reservoir; and
d. controller controlling current flow from the donor reservoir to the body surface, the controller being capable of sending a test current across the donor reservoir to determine the impedance thereof, the controller switching on a drug delivery current flow only after said impedance across the reservoir has fallen below a predetermined condition as the donor reservoir undergoes hydration.

16. The device of claim 15, wherein the donor reservoir comprises a liquid imbibing ester polymer and a cationic drug, the ester polymer having nonesterified carboxyl groups for noncovalently associating with the cationic drug.

17. A method of preparing an iontophretic drug delivery device, comprising: hydrating a hydratable reservoir in an iontophretic drug delivery device by providing a liquid to said hydratable reservoir, sensing impedance across the hydratable reservoir, monitoring the impedance until the impedance has reached a predetermined condition, and not providing more of the liquid to the hydratable reservoir after the impedance has reached a predetermined condition.

18. The method of claim 17 comprising providing a test current between a donor electrode and a monitoring electrode to determine whether the impedance of the donor reservoir has fallen below a predetermined condition, said donor electrode and monitoring electrode electrically communicating through the hydratable reservoir, said test current being inadequate for electrotransport drug delivery.

19. The method of claim 17 comprising providing a controller that acts on information on the impedance across the donor reservoir and automatically activates to enable drug delivery current flow after the impedance across the reservoir has reached a predetermined condition.

20. The method of claim 17 comprising providing the monitoring electrode to contact the donor reservoir and providing a counter electrode and a counter reservoir to contact the body surface to complete current flow path, the counter electrode and the monitoring electrode being different electrodes.

21. The method of claim 17 comprising providing a counter electrode and a counter reservoir for contacting the body surface of skin to provide complete current flow path, the counter electrode and the monitoring electrode being the same electrode such that impedance between the donor electrode and the monitoring electrode includes impedance of the donor reservoir, skin and the counter reservoirs.

22. The method of claim 17 comprising providing a controller that acts on information on the impedance across the donor reservoir and physically connecting an impedance monitoring circuitry to the controller to provide electrical communication on the impedance.

23. The method of claim 17 comprising displaying an audio or light signal when the impedance being monitored has fallen below a predetermined level or when the electrotransport drug delivery current flow starts.

24. The method of claim 17 comprising including a liquid imbibing ester polymer and a cationic drug in the donor reservoir.

25. The method of claim 17 comprising including a liquid imbibing ester polymer and a cationic drug in the donor reservoir, the ester polymer having nonesterified carboxyl groups for noncovalently associating with the cationic drug.

26. The method of claim 17 comprising including a liquid imbibing ester polymer and a cationic drug in the donor reservoir, the liquid imbibing polymer being formed by esterification between hydroxyalkyl polymer and one of polyacrylic acid polymer and polymethacrylic acid polymer.

27. A method of making a device for iontophretic drug delivery, comprising: providing a prehydration device comprising a pair of electrode assemblies, at least one of said electrode assemblies having a donor electrode and a donor reservoir for containing an ionic drug to be iontophoretically delivered, said donor reservoir being hydratable and upon hydration becoming applicable on a body surface for iontophoretic delivery, the donor reservoir in electrically communication with a monitoring electrode at a location different from the donor electrode;
providing electrical communication by a monitoring circuitry to the donor electrode and the monitoring electrode for sensing impedance in the donor reservoir;
providing a liquid to the donor reservoir for hydration; and
sensing impedance in the hydratable reservoir before enabling the device to deliver therapeutic drug delivery current.

28. A method of iontophretic drug delivery, comprising: hydrating a hydratable reservoir by providing a liquid thereto, sensing impedance across the hydratable reservoir, commencing iontophoretic drug delivery after said impedance has reached a predetermined condition.
